(19) United States
(12) Patent Application Publication
(10) Pub. No.: US 2019/0275332 A1
(43) Pub. Date: Sep. 12, 2019

(54) Interactive Virtual Assistant System and Method for Neural Stimulation Therapy

(71) Applicant: Lumbrera LLC, Bloomington, IL (US)

(72) Inventors: David Leonardo Cedeno, Normal, IL (US); Ricardo Vallejo, Bloomington, IL (US); William J. Smith, Bloomington, IL (US)

(21) Appl. No.: 16/298,198

(22) Filed: Mar. 11, 2019

Related U.S. Application Data
(60) Provisional application No. 62/640,982, filed on Mar. 9, 2018.

(51) Int. Cl. A61N 1/36; G10L 15/22; G10L 15/26; G10L 13/04

(52) U.S. Cl. CPC A61N 1/36139; G10L 13/043; G10L 15/265; G10L 15/22

(57) ABSTRACT

An Interactive Virtual Assistant for patients using a neural stimulation system utilizes a processor, speaker, and microphone to actively interact with a patient and address patient needs during neural stimulation therapy and to generate meaningful recommendations and alerts for the patient.

### FIELD OF THE INVENTION

The disclosure relates to a system and method of easing stimulator use and integrating care by means of an Interactive Virtual Assistant for neural stimulation therapies.

### BACKGROUND OF THE INVENTION

The modern aspects of the field of neurostimulation for pain control derive from the seminal paper by Melzack and Wall (Pain Mechanisms: A New Theory, Science, 1965) that formulated the gate control theory for modulation of pain signals with electrical stimulation fields arranged in the afferent pathway. As the field has progressed, many advances have been introduced to the devices, at the level of the stimulation programs used, hardware connectivity (leads, anchors), hardware capabilities (implantable, rechargeable, miniaturization), and implantation techniques. Recent innovations have centered in both the programming device utilized by clinical staff to activate and optimize initial stages of therapy, and the remote controller used by patients on a daily basis to adjust therapy. For example, neurostimulators and their corresponding programmers or remote controllers formerly required hardwired connections. However, with the advent of Bluetooth and RFID technology, neurostimulators have been detached from their programming and controlling units, allowing for easier use by patients. Additionally, more control has been placed in the patient's hands by allowing the patient to optimize available therapy programs. Currently, a patient is provided with a remote controller unit that becomes a permanent companion during their neurostimulation therapy. This device allows patients to switch between therapy programs pre-entered by the physician, and modulate certain therapy parameters, such as the amplitude of electrical stimulation, and even select for certain specified features such as adaptive stimulation capabilities that automatically adjusts stimulation strength based on the patients' body position. While these adaptations have increased patient's sense of control over their own therapy, they have made little headway in easing patient's common frustrations with therapy and fail to improve integration of the healthcare team.

Generally, patients who experience a reduction of the therapeutic benefit must first attempt programming adjustments on their own by adjusting stimulation parameters such as pulse amplitude, width, and/or frequency, or shifting to a separate therapy program that stimulates a different location of the neural structure all together. While efforts have been made to make this process as accessible and simple as possible, many patients still end up frustrated. Disheartened patients have three options: leaving the program as is with subpar stimulation, turning their neurostimulators off, or contacting a representative of the neurostimulator company. Patients who choose to shut off their stimulators often present to the clinic for follow up visits angry and impatient, demanding the physician to fix the issue. Physicians, in this instance, have only as much information as the patient can provide them about what was wrong with the neurostimulator and when therapy began to fail. Most neurostimulation companies have a network of representatives that are available to assist patients so this situation does not arise often. However, this solution also has its pitfalls. Representatives are usually busy, juggling a multitude of patients and actively attending clinics and implantation surgeries. As such, they are often over-burdened and unavailable to attend immediately to patients who are in desperate need. Once they are able to communicate with patients, representatives have limited access to information that can be used for assisting patients on optimizing their neurostimulation therapy. The primary contact method is a phone call where the representative provides verbal instructions to patients on how to address the problem by assisting the patient in navigating the remote controller. If this interaction is unsuccessful, the representative will schedule an appointment to reprogram the patient at the physician's office. In both of these scenarios, the representative may run through the multiple therapy programs already entered into the patient's neurostimulator. The representatives, however, may have no recollection of the number of programs a patient have available nor for what each of program is optimized, leaving patients and representatives to play an educated guessing game to improve the therapy. When all these facts are considered, it is easy to understand how patients may become frustrated with their neurostimulation treatment.

In addition, many of the remote controllers used by patients in neurostimulation therapy lack simple functionalities such as voice-based notification or alerts that warn patients of certain important conditions such as low battery levels. Many remote controller also fail to optimally take advantage of biofeedback capabilities of the implantable neurostimulation units. For example, there are spinal cord stimulation systems equipped with a gyroscope and accelerometer that can track patient's movements and position throughout the day. Such data may recorded and reported, and may be useful to provide feedback to patients when they are trying to optimize their therapy.

In summary, although current clinical programmers and patient remote controllers have come a long way since their inception, the latest still fail to address many of the issues faced by patients regarding the maintenance and optimization of their neurostimulation therapy.

Accordingly, a need exists for an interactive remote controller to ease patient frustration and streamline many point-of-care applications of neurostimulation.

A further need exists for an interactive remote controller that alerts the patient to conditions such as low battery levels.

A still further need exists for an interactive remote controller that utilizes biofeedback capabilities of the neurostimulation unit with which the remote controller is associated.

### SUMMARY OF THE INVENTION

The disclosed system provides interactive companionship through a virtual assistant integrated with the patient remote controller device. Disclosed is a system and method for an intelligent interactive virtual assistant (IVA) for integration to neurostimulation remote controllers (hereafter also referred to in the figures as the “StimBuddy” IVA application) for use with a neural stimulation systems. The disclosed system and a method of use facilitates use of neural stimulation therapies, including, but not limited to, spinal cord stimulation (SCS), peripheral nerve stimulation (PNS), dorsal root ganglion stimulation (DRG Stim), and deep brain stimulation (DBS).

The IVA device may include any of a central processor unit (CPU), microprocessor unit (MPU) or microcontroller (MCU), a display, a speaker, and/or a microphone to facilitate verbal as a well as visual interaction with the patient. The disclosed IVA device utilizes speech recognition and text to speech software to create an interactive virtual environment for the patient.

In embodiments, the IVA device utilizes the CPU already included in the patient remote controller. In another embodiment, the IVA device uses a separate CPU to reduce burden on the CPU used for controlling stimulation. In another embodiment, the IVA device uses a remote CPU to which the IVA device is operatively coupled. In all embodiments, any of the CPU, MCU, or MPU may be integrated within the remote controller unit housing and, thus, will appear as part of the patient remote controller, or may be remotely linked thereto via wireless protocols.

In embodiments, memory associated with the IVA device stores a library of functional questions to ask patients. These questions include pain-state level questions such as, “What is your current pain level?”, “What is your current pain quality?”, “Where is your pain located?”, and any other pain-state question. The library may also include questions related to activity and disability states, such as “How have you been sleeping?”, “Have you had problems with personal care or dressing?”, and any other similar questions. The library may also include questions related to the emotional well-being of the patient, given the high incidence of depression and anxiety in chronic pain patients. The questions outlined herein are not meant to limit functionality and the library can include any questions deemed fit for improving patient's therapeutic experience and easing their frustration. As described elsewhere herein, such phrases intended to evoke interaction with the patient may be stored in text format for optimal storage efficiency and synthesized into audio by text to speech conversion algorithms associated with the IVA device. The speech to text and text to speech software may comprise one or more open source software modules already developed, modifications to existing software, licensure of existing STT softwares, or development of a unique STT software for the IVA device described herein.

In embodiments, the IVA device allows for hands free control of the remote control by the patient and for biofeedback from both the patient and from the neurostimulator. In another embodiment, this biofeedback functionality is integrated with the question libraries associated with the IVA device to improve question relevance. The IVA device CPU may analyze motion and position data from gyroscopes and accelerometers embedded in the neurostimulator, resulting in a question that reflects the patient's activity level such as, “I noticed you haven't been moving much lately, how are you feeling?” or “I noticed you have been laying down for several hours, how would you rate your feeling of sadness/depression today?”

In embodiments, the IVA device records patient responses through speech recognition and speech to text algorithms. The patient responses may include answers to aforementioned questions from the IVA device library and program alterations. These responses may be used to inform and improve questions and suggestions made by the IVA device.

In embodiments, the IVA device will respond to patient requests upon detection of an “activation phrase.” Upon activation, the IVA device listens for keywords related to its functionalities. These keywords may include phrases related to the pain state, disability/activity level, emotional state, reprogramming needs, etc. Upon detection of a keyword, the IVA device will respond with either a follow up question or execution of a certain program that addresses the patient's needs. In embodiments, these program responses may be informed by previous responses that have been stored in the IVA device and used to adapt the algorithm.

In embodiments, the IVA device updates the patient with alerts regarding important information pertaining to stimulation settings or states, such as the status of battery charge, and/or a reminder of the appointment with the pain physician.

In embodiments, the IVA device creates an organized data structure of stored patient data. The report may contain information on the pain states, programming changes, disability/activity states, and emotional states with time stamps. This information may be transferred to a device in the physician's office, such as a tablet or computer, for the physician and/or company representative to review. This functionality offers physicians, representatives, and researchers an unparalleled level of insight into how patients interact with stimulators and provides a large potential for improvement of neurostimulation therapies.

According to one aspect of the disclosure, a virtual assistant system for use with a neurostimulation signal generator, controllable by at least one selectable stimulation signal parameter, comprises: a controller device operatively couplable to the signal generator, the controller device comprising a user interface operable to communicate one of visual and audio data; a memory operatively couplable to the controller device and operable to store biofeedback data; and a recommendation engine executable on a processor and operatively couplable to one of the controller device and the signal generator and further operable to receive biofeedback data from one of the controller device and the signal generator, wherein the recommendation engine is further operable to provide a recommendation through the user interface to change the at least one selectable stimulation signal parameter in response to the biofeedback data. In one embodiment, the biofeedback data storable in the memory comprises data representing any of a location pain of a patient, a level of pain of a patient, a stimulation signal parameter identifier, and time identifier. In other embodiments, the memory is further operable to store one of keywords and phrases receivable through the user interface of the controller device or a plurality of predefined keywords or phrases presentable through the user interface of the controller device.

According to another aspect of the disclosure, a method for interacting with a neurostimulation signal generator having a controller device operatively couplable to the signal generator, the method comprises: A) receiving biofeedback data from one of the controller device and the signal generator; B) providing a recommendation through a user interface associated with the controller device to change at least one selectable stimulation signal parameter associated with the signal generator in response to the biofeedback data, wherein the recommendation provided through a user interface is based on a plurality of biofeedback data received from one of the controller device and the signal generator. In one embodiment, the biofeedback data comprises data representing any of a location pain of a patient, a level of pain of a patient, a stimulation signal parameter identifier, and time identifier. In other embodiments, the biofeedback data comprises one of keywords and phrases received through the user interface of the controller device. In other embodiments, the biofeedback data comprises positional data from the signal generator.

According to still yet another aspect of the disclosure, a computer program product for use with a processor, the computer program product comprising a non-transitory tangible medium having computer executable instructions embed thereon which when executed perform a method for interacting with a neurostimulation signal generator, the method comprising: A) receiving biofeedback data from one of a controller device and a signal generator; and B) providing a recommendation through a user interface associated with the controller device to change at least one selectable stimulation signal parameter associated with the signal generator in response to biofeedback data, wherein the recommendation provided through a user interface is based on a plurality of biofeedback data received from one of the controller device and the signal generator.

### DESCRIPTION OF FIGURES

The various features and advantages of the present invention may be more readily understood with reference to the following detailed description taken in conjunction with the accompanying drawings, wherein like reference numerals designate like structural elements, and in which:

FIGS. 1A-B illustrate conceptually the components of a neurostimulation system, in accordance with the disclosure;

FIG. 2 is an exemplary block diagram of a signal generator device, in accordance with the disclosure;

FIG. 3 is an exemplary illustration of the physical embodiment of an IVA device, in accordance with the disclosure;

FIG. 4 is an exemplary flow chart representing a “pain detection” algorithm, where the patient initiates an interaction with the IVA device through an activating key word or phrase, in accordance with the disclosure;

FIG. 5 is an exemplary flow chart representing a “pain query” algorithm, where the IVA device initiates an interaction with the patient based on a certain precipitating event, in accordance with the disclosure;

FIG. 6 is an exemplary flow chart representing a “system alert” algorithm, wherein the IVA device detects a certain event, in accordance with the disclosure; and

FIG. 7 is a flow chart representing a “contact representative” algorithm, wherein the IVA device initiates contact with a third party, in accordance with the disclosure.

### DETAILED DESCRIPTION

The system and methods described herein will embody many potential forms and techniques for implantation of a system capable of relieving pain associated with various chronic pain states (e.g. nociceptive, inflammatory, neuropathic pain, or combination thereof). The specific design of the system, including the IVA system and device described herein, is a guideline for understanding the system, but it will be obvious to the skilled in the art that it shall not limit the range of its description.

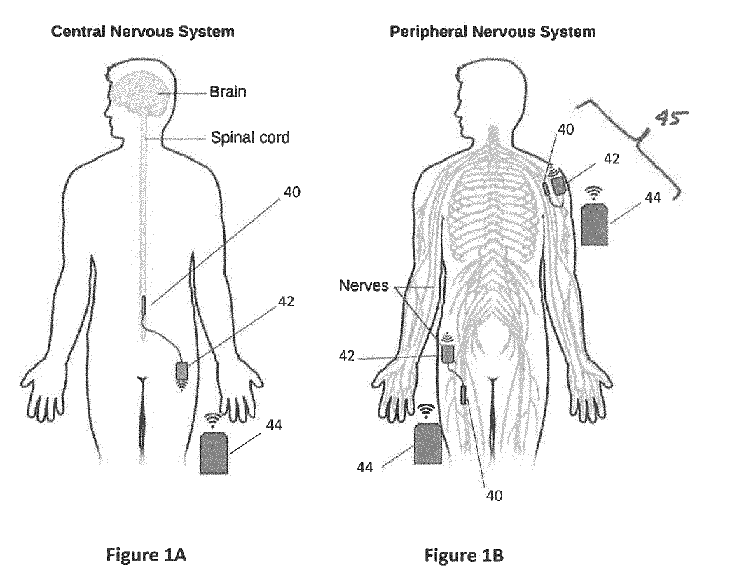

FIGS. 1A-B and 2 illustrate conceptually embodiments of a neural stimulation system 45 that may be utilized with the interactive virtual assistant device disclosed herein. In embodiments, as illustrated in FIGS. 1A-B, the system 45 comprises electrical leads 40 containing an array of electrodes, a neural stimulation device 42 and a remote control device 44 into which all or part of the interactive virtual assistant functionality described herein may be incorporated. Any of the functions described herein may be located within any of the elements 40, 42 and 44, except where specified otherwise.

Device 42 may be may be hermetically sealed in a housing made of a durable biocompatible material, such as stainless steel or titanium, and has an output interface for establishing electrical connection with electrode(s) implemented within leads 40 that deliver the correction signals to glial cells and neurons and communicate with remote control device 44 through appropriate connectors. Device 42 is electrically coupled to electrical leads 40, each of which may be implemented with at least one or more electrode contacts. In an embodiment, a pair of leads is coupled to the device 42 using appropriate connectors. In another embodiment, a single lead implemented with an array of electrodes can be used. A neural stimulation device suitable for use as device 42 in the neural stimulation system 45 described herein is described in United States Patent Application Publication US 2018/0185645 A1, Ser. No. 15/860,117, by Vallejo et al, entitled “SYSTEM AND METHOD OF PAIN RELIEF BASED ON FREQUENCY BASED ANALYSIS OF TEMPORAL NONCICEPTIVE SIGNALS” filed Jan. 2, 2018, the subject matter of which is incorporated herein by this reference for all purposes. In addition, either device 42 or IVA device 10 may include an inertial measurement device 26, such as a multi-axis gyroscope and accelerometer for tracking patient activity and orientation, as described herein.

Within the neural stimulation system 45 exists the interactive virtual assistant system 110, most components of which may physically reside in housing of the remote control device 44, but may also be associated with IVA device 44 even though located remotely therefrom, as described elsewhere herein. Except for the user interface, any of the functions described herein with reference to IVA device, may be located within of neural stimulation device 42, remote controller 44, or in a separate remotely located device to which either of neural stimulation device 42 or remote controller 44 are operatively coupled.

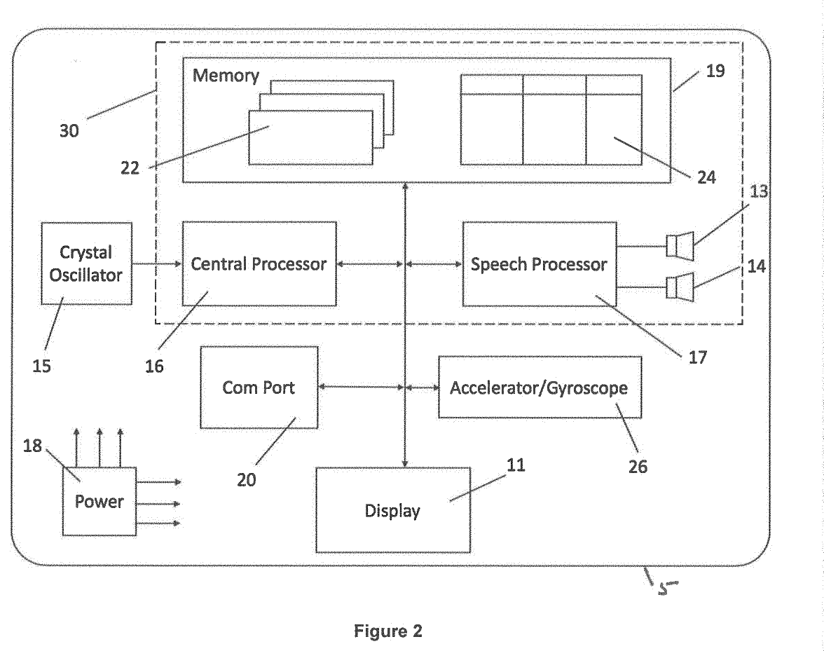

In one embodiment, as illustrated in FIG. 2, IVA system 5 may comprise a processing module 16 associated with a neural database/memory 19 and a communication port 20 for transmitting data to and from neural stimulation device 42 or other device such, as a mobile device or remote computer, via a transceiver, e.g. a Bluetooth transceiver, a Bluetooth transmitter, a radio-frequency transceiver, a radio-frequency transmitter, a WiFi transceiver, and a WiFi transmitter, via any protocol including serial communication device pursuant to RS232 standard, Bluetooth, or other communications protocol.

In embodiments, central processing module 16 may be implemented with any number of small, medium or large scale electrical processing or logic components, such as those described herein, and may be embodied with a small footprint. In embodiments, processor module may be implemented in an integrated circuit package and may comprise any of one or more microcontrollers, microprocessors, a programmable logic controller (PLC), a field programmable gate array (FPGA), or an application-specific integrated circuit (ASIC), collectively referred to hereafter as the processor. The central processing module 16 may be implemented with a microprocessor integrated circuit or may comprise reduced functionality small-scale logic, but, in either implementation, includes a wireless transceiver functionality that enables bidirectional wireless communication of information with an external programmer unit (not shown). The neural database/memory 19 associated with module 16, may be implemented with any combination either RAM or ROM memory, and is used to store a processing program, executable by central processing module 16, which speech processing module 17. The central processing module is able to store and retrieve information from associated memory, as necessary. The power source 18 may comprise a rechargeable battery and electronic circuity that distributes power from the battery to all the other components in device 44. A protocol may be provided for operating the device 44 in a low power mode and selectively initiating the processing modules 16 or 17 to a higher-power state with increased data retention in relation to the acquired signal. The speech processing module 17 may comprise electronic circuitry that allows any execution of speech recognition programs, and/or text to speech or speech to text synthesis programs, as well as programs for generating more traditional audio queues associated with user interfaces for alarms and timers, etc.

In one embodiment, speech module 17 is controlled by or operates in conjunction with module 16 or external programmer unit. Note, module 17 may be implemented with analog or digital circuitry or a combination thereof. In one embodiment, all or most of the functional elements of module 17 may be fabricated on a single integrated circuit chip including a processing logic and associated memory, and one or more digital oscillators. Alternatively, the digital oscillators may be replaced with wave tables having stored therein mathematical descriptions of various waveform data values which are convertible into analog signals using a digital to analog converter. Such wavetables may be stored in processor module 17 or memory module 19. In embodiments the various components of IVA system 5 may communicate as illustrated in FIG. 2 or over a central bus internal to device 44 or may have dedicated direct connections therebetween, or any combination thereof.

In embodiments, either of devices 42 or 44 may be programmed by a clinician using software that allows control of all the aspects of the system 45. The software may be accessible in a computer-based interface called the Clinician Programmer (CP) software. The software may be implemented with wireless communication protocols for remote access of the device 42. The CP software enables the clinician to communicate with module 16 and 17 and memory 19 to define a set of parameters, e.g. any of keyword or phrases in the speech library in either text or waveform format. Such defined parameter sets may be stored as one or more configuration programs in memory 19 associated with and transmittable to device 42 via telemetry logic for control of module 117. The CP software may enable the clinician to further define which parameter the patient may control with the remote device 44 and to define any limits on such parameters. The IVA system 5 allows for setting and storing additional configuration programs deemed necessary for the clinician and according to the storage capacity of the memory module 19.

FIGS. 1A-B illustrate conceptually implantation locations of neural stimulation system 45, both in the central nervous system through a spinal cord implantation and in the peripheral nervous system through major peripheral nerves, such as the sciatic and brachial. In embodiments, the system 45 comprises electrical leads or cuffs 40 containing an array of electrodes, device 42 and optional remote control device 44. The leads 40 are placed surgically or percutaneously in the epidural or subdural space of the cervical, lumbar or thoracic spinal cord of a patient, as illustrated in FIGS. 1A-B, and connected to the signal-compensating device 42, which may be implanted in an appropriate area of the body or positioned outside the body. Lead 40 may be connected to device 42 via wireless transmission protocols. In still other embodiments, the signal-compensating device 42 may be implanted in an appropriate area of the body or worn outside the body and communicate wirelessly with a user remote control 44 which communicates wirelessly with signal-compensating device 42.

The electrical leads 40 may be placed surgically or percutaneously, using fluoroscopic, ultrasound or other imaging technique guidance, in the proximity of the DRG or intraspinal nerve roots. In embodiments, the leads 40 and/or device 42 may be placed in the proximity of the peripheral nervous system for nerves extending to the limbs, including the sciatic and femoral nerves or any of its branches in the lower limb and the brachial nerve or any of its branches in the upper limb, as illustrated in FIGS. 1A-B. In order to localize the nerves, several imaging guiding techniques, including, but not limited to, ultrasound, fluoroscopy, magnetic resonance images, computer tomography, electromyography, etc. Alternatively, sympathetic or parasympathetic fibers may be stimulated to control other conditions included, but not limited to headaches and migraines (occipital nerves), facial pain (facial nerves, sphenopalatine ganglion, trigeminal nerves), complex regional pain syndrome (stellate ganglion, lumbar sympathetic nerves, etc.), abdominal pain (splanchnic nerves, celiac plexus, superior hypogastric ganglion, impar ganglion), These methods can be extended for other peripheral nerves in the body.

In embodiments, instead of lead 40, a stimulation lead (not shown) may be implanted in the intervertebral disk for stimulation of the afferent supraspinal tract. Such electrical stimulation lead may be inserted in the posterior side of an intervertebral disc in order to stimulate the spinal cord, herein called Transdiscal Spinal Cord Stimulation. In embodiments, another lead is placed through the opposite superior articular process space and is used to pull the lead through for placement. The intradiscal lead may be placed in any of the intervertebral discs between vertebral levels T8 and L2, wherein the superior articular process space is large enough for access and the spinal cord remains intact prior to its split into the cauda equina. The placement of the lead in the intradiscal location allows for stimulation of the supraspinal tract, where afferent fibers from the periphery travel to carry information to the brain.

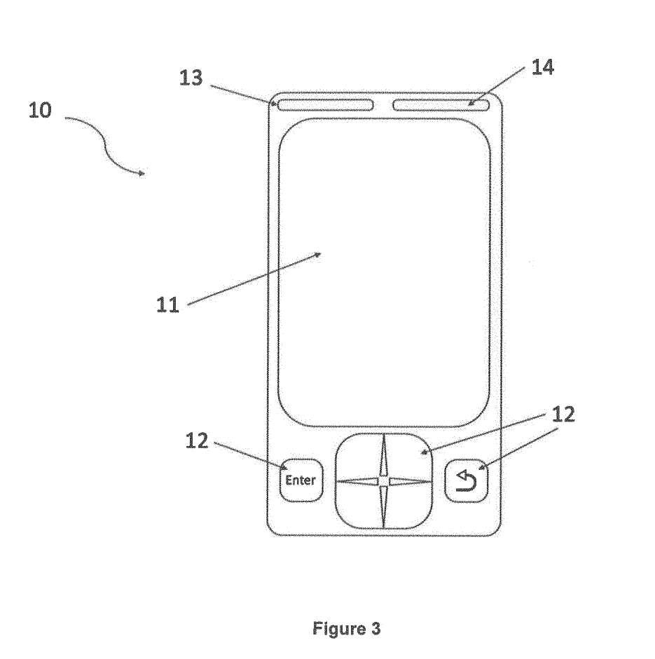

FIG. 3 illustrates one implementation of the remote device into which the Interactive Virtual Assistant (IVA) system 5 disclosed herein may be implemented, referred to hereafter as IVA device 10. The IVA device 10 comprises a display 11 to present pertinent information regarding programs and battery power and a set of controls 12, e.g. Enter and Back and directional navigation buttons, through which the user may interact with the IVA device 10. Note the controls 12 may be implemented either physically hardwired or rendered virtually as part of a graphic user interface rendered for viewing on the display 11. The device 10 further comprises a microphone 13 and at least one audio transducer 14, e.g. a speaker, to allow for two-way communication. In embodiments, audio transducer 14 may act as both microphone and speaker.

In one implementation, speaker 14 and microphone 13 may be integrated into an existing patient remote controller device by upgrading one or more of the integrated circuits (ICs) components within existing remote controller circuitry to be controlled and programmed through the existing CPU in the remote device. Alternatively, the audio transducer can be controlled through a CPU remote from the controller device 44, allowing for increased storage space for data and processing power while only marginally increasing the physical space needed within the housing of IVA device 10.

In embodiments where the IVA device 10 uses a separate CPU, the IVA device uses a CPU capable of running software within a Python, C, Java environment, or any other available or custom-made software designed for programming commands that provide functionality to the IVA system 5 within the context of neural stimulation system 45. The IVA device CPU has input connections to a communication port 20 of device 10. This connection allows sending information between the other components of the IVA system depending on the implementation and remote controller device, as well as receiving information from other components in the neural stimulation system 45. Relevant information may include data pertaining to patient position and movement as generated analyzed by hardware included in either of devices 42 or 44, as well as program state, and battery level. In embodiments, such information may be transmitted to the IVA device 44 as serial data and processed by the CPU assigned to the IVA device 10. In another embodiment, the CPU of the remote controller device sends information already processed and encoded to the CPU assigned to the IVA neural stimulation system. In both embodiments, the information is actively analyzed to determine specific values that trigger a program execution.

In one embodiment, any of components 15, 16, 17, 18, 19 and 20 may be implemented within device 44, in a single integrated circuit package or multiple integrated circuit packages.

In embodiments, the IVA device 10 may contain a clock capable of keeping time in synchronicity with the geographically relevant time zone, as can be accomplished by a quartz oscillator chip 18 that has a fundamental frequency of n Hz and a counter. When that counter reaches 86400*n Hz, the counter will reset to zero. During programming, the representative of the neuro-stimulator company or the patient will be able to input the current time, allowing the counter to correlate its counter value with a 24-hour clock time. In embodiments, the user will be able to use a key-phrase such as “time settings” to adjust the time.

In embodiments, the IVA device 10 will respond to a detected “triggers” or audio queues having audio signals similar to natural language words or sounds, including activation phrase such as a “Hey Buddy”, “Buddy”, or any other preprogrammed or customizable audio queue. Such triggers may also include any of natural language words, sounds, keywords or phrases such as “ow”, “hurts”, or “aches” from the pain-state library associated with the IVA system 5. Triggers may also include data from the neurostimulator device 42 such as battery level or motion and position data. For example, if the patient's positional data, e.g. gyroscopic and motion acceleration data, has remained steady or substantially the same for several hours during the day, the IVA system 5 may recognize this as a trigger for IVA device 10 to activate an action call to the patient. Upon recognition of one of these triggers by IVA system 5, a program or series of programs will be executed to address the patient's needs. This may include responding to the “activation phrase” to discern patient need, initiating a question sequence from one of the IVA device libraries, or delivering an alert or prompt to the patient.

In embodiments, the IVA device 10 allows for storage of patient responses in local or remote memory to facilitate analysis the patient's interaction with IVA system 10 and to improve IVA device 10 response(s) to requests. When the patient is asked a question from a specific neural library 22, such as their pain level, the response is analyzed by module 17 using speech to text conversion algorithms, and will be appended to the end of a data structure such as a data matrix 24, storable in memory 19, where a first column of the data matrix contains data representing a pain number, e.g. from a scale of 1 to 10, and a second column contains a time stamp of when such indication was received by IVA device 10. Such informational data matrixes may be used to store information that can be analyzed to recommend a program change, if necessary. If the patient requests a program change due to a specific level of pain, upon finding the most beneficial program, the IVA device 10 will access the matrix for that program. Data identifying the location of pain for which the program was used, the stimulation parameters (pulse frequency, width and amplitude) used, and a time stamp may all be appended to the end of the matrix. This allows the IVA device 10 to store all information entered by the patient. In embodiments, the stored information may also include encoded information rather than raw data, including sleep quality number range, e.g. between 0-10, based on amount of movement during sleep or an activity score range, e.g. from 0-10, based on positional changes and accelerometer data.

In one embodiment, the data matrix 24 of stored patient responses and biofeedback data will additionally incorporate changes in program that occurred as a result. For example, after developing breakthrough pain in their hip the patient changes a parameter of their program and the IVA device will make note of the previous program, updated program, and any other changes that occurred. This continuous tracking of changes to programming in conjunction with biofeedback data will allow for the IVA device to utilize machine learning functionalities to adapt and optimize patient therapy as treatment progresses.

In embodiments, the CPU module 16, memory 19 and speech processor 17 functionally form a recommendation engine 30 which provide the majority of the functionality described herein relative to interactions of the IVA device 10 with a patient.

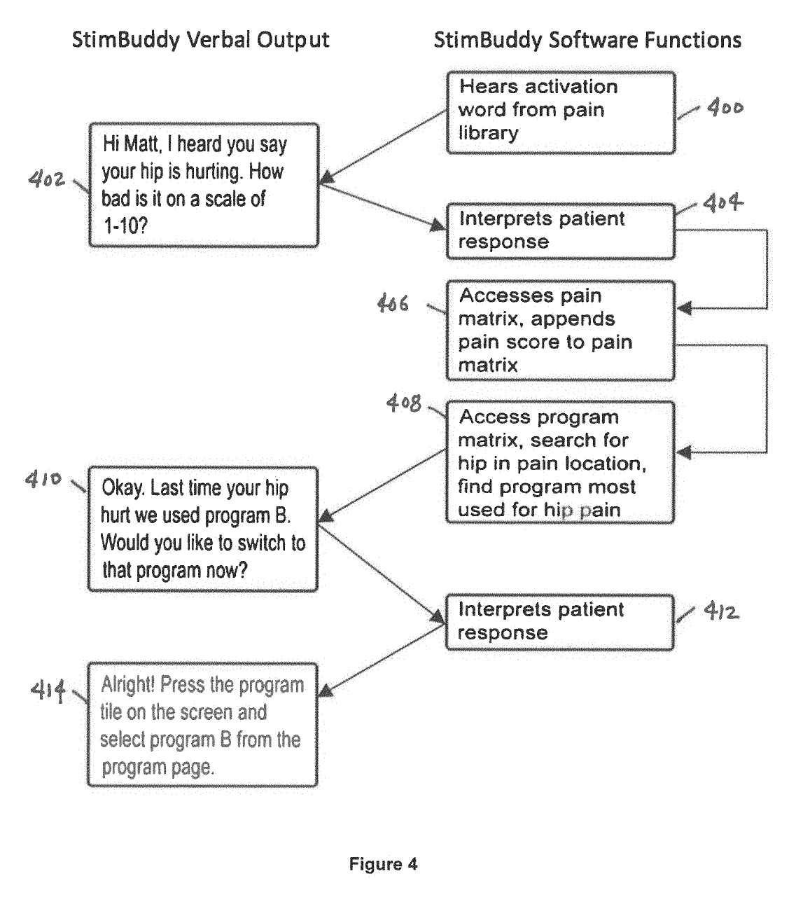

FIGS. 4-7 are flow charts illustrating algorithmic work-flows of the IVA device 10 to ease patient-stimulator interaction.

The IVA device 10 may be programmed to the collected and stored information to suggest changes in therapy programs that may result in an improvement. While current company representatives may not have exact knowledge of which therapy program addresses certain painful areas, IVA device may maintain or have access to several matrices with data representing pain locations, pain intensity, and the therapy program that have been selected by the patient to alleviate the pain. For example, if a patient complains to IVA device of hip pain, the IVA device can pull up the matrices associated with each therapy program and search the location of the word “hip” in the matrix. The therapy program with the most “hip” entries will have its information pulled, including stimulation parameters. This information may then be suggested as a first therapy program for the patient to switch, reducing confusion and guess and check work by patients and representatives.

FIG. 4 is a flow chart representing a “pain detection” algorithm, where the patient initiates an interaction with the IVA device 10 through an activating key word or phrase, as illustrated by process blocks 400 through 414. The workflow example of FIG. 4 illustrates how the IVA device responds in a situation where a patient complains of increased pain in a particular body location. To begin, the IVA device, upon power-up goes, into a low power listening mode and compares any received audio signal with keywords or phrases from the pain-state library, as illustrated by process block 400 and the prompts the patient for additional information using preselected dialogue, as illustrated by process block 402. Such preselected dialogue may be stored as text and converted to speech by the CPU associated with the IVA device using text to speech conversion algorithms. Next, the IVA device interprets the patient response using speech to text conversion algorithms, as illustrated by process block 404, and updates the profile associated with the patient by, for example, accessing the pain patient matrix and appending a new pain score and time stamp, as illustrated by process block 404. The IVA device may continue a back and forth dialogue with the patient to obtain qualifying information about the pain, including, but not limited to, severity, characteristic (i.e. burning, shooting, stabbing, etc.), and location. In the illustrative example, assuming the patient indicated a level of hip pain, the IVA can then access its own data log matrix to identify past instances that match this pain description and the various programs with their specified pain type and pain location identifiers, as illustrated by process block 408. The IVA can then suggest the best program to the patient based on these data, asking if the patient would like to change programs, as illustrated by process block 410. The IVA can continue this interaction to optimize pain relief through reprogramming by interpreting the patient responses, as illustrated by process block 412, and instructing the patient to change programs, as illustrated by process block 414. Once optimization is complete, the program will store this instance of pain detection interaction in its pain log matrix associated with the patient. Additionally, the IVA device is capable of modifying the program identifiers to further specify the type of pain descriptor and pain location for which each program works best.

In embodiments, algorithms associated with the IVA device 10 identify abnormal patterns in biofeedback parameters, such as position and movement as measured by a multi-axis, e.g. 9-axis, gyroscope and accelerometer, which is included in commercially available implantable program generators (IPG) or may be integrated into the IVA device 10 itself. The IVA system queries the patient to discern if the abnormal pattern is linked to any pain or discomfort. In the instance that the abnormality in the biofeedback pattern is related to pain or discomfort, the IVA device can suggest alterations to the stimulation programming to optimize therapy as described herein. Additionally, this algorithm demonstrates the ability of the IVA device to set reminders about altering programming, such as turning on “paresthesia-free” stimulation before bed.

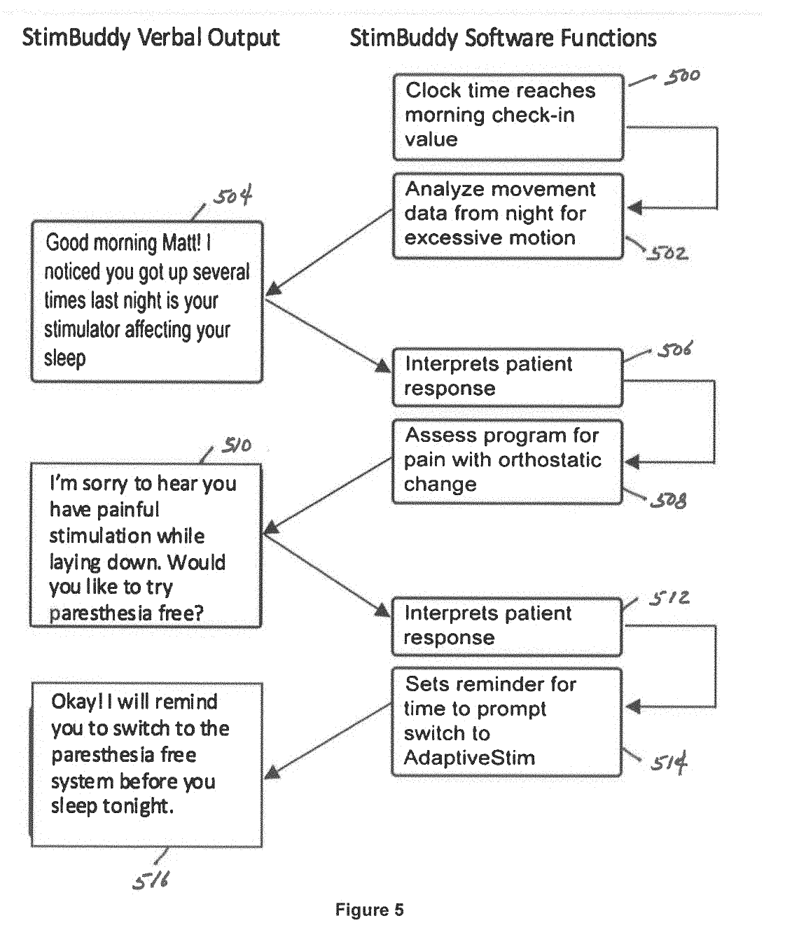

The IVA device 10 may be programmed to ask targeted questions and make targeted decisions. For example, IVA device 44, as a background task, may assess the patient's sleep scores for the past several days to look for a pattern, as illustrated in FIG. 5. If a pattern of low sleep scores is found, IVA device query the patient if their current stimulation program has interfered with their sleeping. Depending on the patient's response, the IVA device may prompt an action command, such as suggesting to the patient a change to another stimulation program, or adjustment of one or more stimulation parameters. Such changes may also be programmed to occur automatically and/or by remote instructions from a care provider.

FIG. 5 is an exemplary flow chart representing a “pain query” algorithm, where the IVA initiates an interaction with the patient based on a certain precipitating event such as a set time or a detection of movement or any combination of other feedback parameters, as illustrated by process blocks 500 through 516. The workflow example of FIG. 5 illustrates how the IVA device utilizes biofeedback from the simulation device 42. The IVA device 10 analyzes the data from the previous night and looks for a threshold value of movement and position, as illustrated by process blocks 500 and 502. The IVA device proceeds to inquire a pain state from the patient, as illustrated by process block 504, interprets the patient response, as illustrated by process block 506, and assess the program for pain with orthostatic change, as illustrated by process block 508. The IVA device may then suggest changes to the current program, as illustrated by process block 510. Again, the IVA device 10 interprets the patient response, as illustrated by process block 512, and set a reminder to prompt a switch in program, as illustrated by process block 514, and the notifies the patient of the upcoming reminder, as illustrated by process block 514.

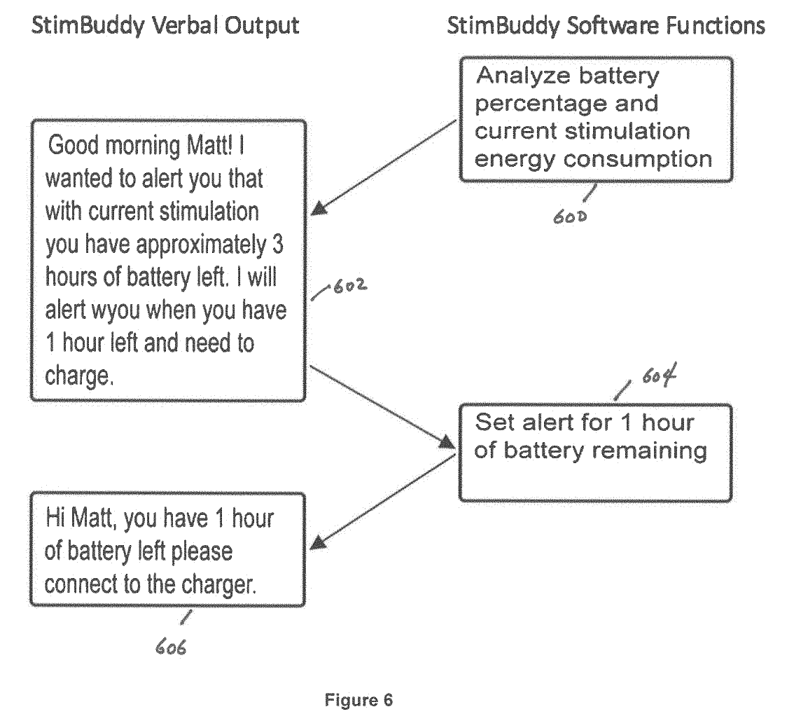

FIG. 6 is a workflow example showing how the IVA device sends alerts to the patient verbally to inform them of important updates regarding the neurostimulation system, as illustrated by process blocks 600 through 606. The workflow example of FIG. 6 illustrates a “system alert” algorithm, wherein the IVA system detects a certain event, such as diminished battery power, and provides an alert to the patient. In this figure, the IVA system detects a battery percentage below a predetermined threshold. The IVA device 10 may notify the patient that under the current program the patient has a certain number of hours remaining until the battery is completely drained. In one embodiment, the IVA device will use the stimulation parameters to calculate the amount of energy transferred per minute. The IVA system is able to perform this calculation through determining the power per pulse of the current program, equal to voltage amplitude multiplied by the current amplitude. The remaining battery power can be divided by the power per pulse to calculate how many pulses the generator can produce. This value can then be divided by the frequency, to determine how many seconds before battery power reaches zero. This calculation will then be compared to the remaining battery level to determine a relatively decent exact battery life.

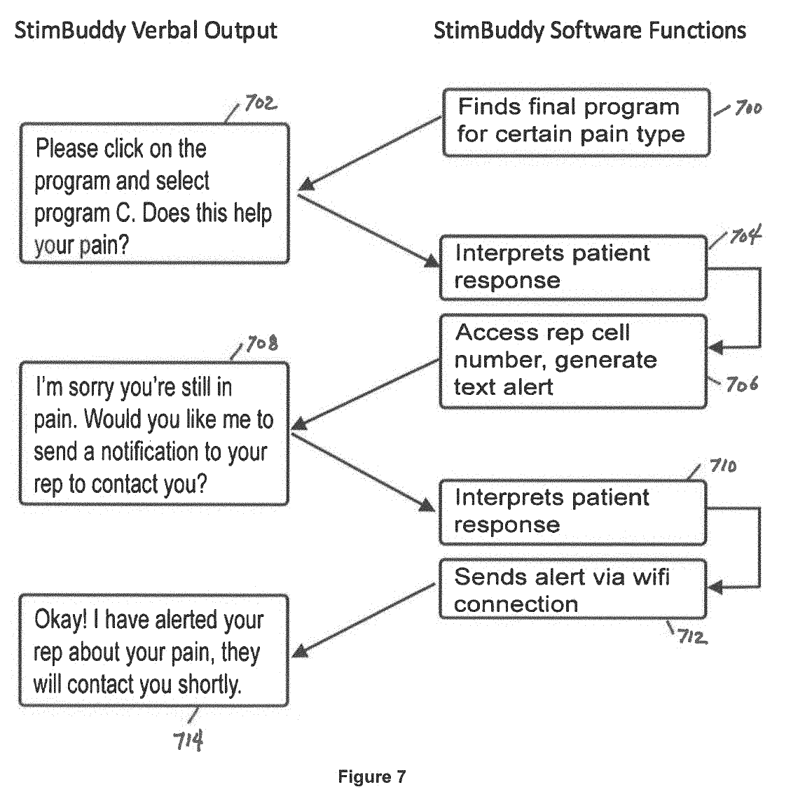

In situations where the patient's efforts to adjust programs with IVA device fails to provide adequate relief, the IVA device 10 can suggest contacting a rep or can send a text, email or other notification to a remote address, such as that of the neurostimulator company representative. This will ease burden on the representatives to make check in calls, while reducing call volume down to only the necessary calls. FIG. 7 is a workflow example showing how the IVA device 10 responds in a situation where patients are unable to obtain relief from their reprogramming attempts, as illustrated by process blocks 700 through 714. The IVA device is able to suggest contacting the neurostimulator company representative or send a notification to provide further assistance in obtaining relief for the patient. The workflow example of FIG. 7 illustrates a “contact representative” algorithm, wherein the IVA system attempts at optimizing patient pain through reprogramming but is unsuccessful. In the instance that the patient states that they would like to contact the representative or the IVA system has exhausted all possible reprogramming options, the IVA will access the contact information of the representative. The IVA system will send an alert to the representative that the patient requires reprogramming and the representative can coordinate with the patient to schedule this.

In the disclosed system, biofeedback data may comprise any data or information that is stored by, presented by, generated by, recorded by, received by, transmitted by or communicated with neural stimulation system 45 or IVA system 5, including, but not limited to, data or information representing any of a location pain of a patient, a level of pain of a patient, a stimulation program identifier, a stimulation signal parameter identifier or value, a time identifier, counter values or any communication or network address.

It will be appreciated that any of the aspects, features and options described in view of the methods apply equally to the system and devices described herein. It will be understood that any one or more of the above aspects, features and options as described herein can be combined. The techniques described in this disclosure may be implemented, at least in part, in hardware, software, firmware or any combination thereof. Such hardware, software, and firmware may be implemented within the same device or within separate devices to support the various operations and functions described in this disclosure

It will be obvious to those reasonably skilled in the relevant arts that modifications to the apparatus and process disclosed herein may occur, including design and implementation of various algorithms for improved patient interaction and therapy optimization, without parting from the true spirit and scope of the disclosure.

What is claimed is:

1. A virtual assistant system for use with a neurostimulation signal generator controllable by at least one selectable stimulation signal parameter, the virtual assistant system comprising:
a controller device operatively couplable to the signal generator, the controller device comprising a user interface operable to communicate one of visual and audio data;
a memory operatively couplable to the controller device and operable to store biofeedback data;
a recommendation engine executable on a processor and operatively couplable to one of the controller device and the signal generator and further operable to receive biofeedback data from one of the controller device and the signal generator;
wherein the recommendation engine is further operable to provide a recommendation through the user interface to change the at least one selectable stimulation signal parameter in response to the biofeedback data.

2. The virtual assistant system of claim 1 wherein signal generator is controllable by a plurality selectable stimulation signal parameter.

3. The virtual assistant system of claim 2 wherein the plurality selectable stimulation signal parameters are selectable through the user interface of the controller device.

4. The virtual assistant system of claim 1 wherein biofeedback data comprises data indicating a location or level of pain of a patient.

5. The virtual assistant system of claim 1 wherein the signal generator generates biofeedback data comprising positional data of a patient.

6. The virtual assistant system of claim 1 wherein the controller device further comprises a housing and wherein one of the memory and recommendation engine are disposed in the housing.

7. The virtual assistant system of claim 1 wherein the controller device further comprises a housing and wherein one of the memory and recommendation engine are not disposed within in the housing.

8. The virtual assistant system of claim 1 wherein the biofeedback data storable in the memory comprises data representing any of a location pain of a patient, a level of pain of a patient, a stimulation signal parameter identifier, and time identifier.

9. The virtual assistant system of claim 1 wherein the memory is further operable to store one of keywords and phrases receivable through the user interface of the controller device.

10. The virtual assistant system of claim 1 wherein the memory is further operable to store a plurality of predefined keywords or phrases presentable through the user interface of the controller device.

11. The method for interacting with a neurostimulation signal generator having a controller device operatively couplable to the signal generator, method comprising:
A) receiving biofeedback data from one of the controller device and the signal generator;
B) providing a recommendation through a user interface associated with the controller device to change at least one selectable stimulation signal parameter associated with the signal generator in response to the biofeedback data,
wherein the recommendation provided through a user interface is based on a plurality of biofeedback data received from one of the controller device and the signal generator.

12. The method of claim 11 wherein the biofeedback data comprises data representing any of a location pain of a patient, a level of pain of a patient, a stimulation signal parameter identifier, and time identifier.

13. The method of claim 12 wherein the biofeedback data comprises one of keywords and phrases received through the user interface of the controller device.

14. The method of claim 11 wherein the biofeedback data comprises positional data from the signal generator.

15. A computer program product for use with a processor, the computer program product comprising a non-transitory tangible medium having computer executable instructions embed thereon which when executed perform a method for interacting with a neurostimulation signal generator, the method comprising:
A) receiving biofeedback data from one of a controller device and a signal generator; and
B) providing a recommendation through a user interface associated with the controller device to change at least one selectable stimulation signal parameter associated with the signal generator in response to biofeedback data,
wherein the recommendation provided through a user interface is based on a plurality of biofeedback data received from one of the controller device and the signal generator.

16. The computer program product of claim 15 wherein the biofeedback data comprises data representing any of a location pain of a patient, a level of pain of a patient, a stimulation signal parameter identifier, and time identifier.

17. The computer program product of claim 16 wherein the biofeedback data comprises one of keywords and phrases received through the user interface of the controller device.

18. The computer program product of claim 15 wherein the biofeedback data comprises positional data from the signal generator.
